(19) United States
(12) Patent Application Publication
(10) Pub. No.: US 2023/0285236 A1
(43) Pub. Date: Sep. 14, 2023

(54) INFUSION PUMP DEPLOYMENT SYSTEMS AND METHODS

(71) Applicant: Cam Med Inc., West Newton, MA (US)

(72) Inventors: Renwei Liu, Woburn, MA (US); Eduardo Luis Latouche, Chelsea, MA (US); John Richard Bussiere, Littleton, MA (US)

(73) Assignee: Cam Med Inc., West Newton, MA (US)

(21) Appl. No.: 18/014,862

(22) PCT Filed: Jul. 6, 2021
(86) PCT No.: PCT/US2021/040450

§ 371 Date: Jan. 6, 2023

Related U.S. Application Data
(60) Provisional application No. 63/049,024, filed on Jul. 7, 2020.

(51) Int. Cl. A61J 1/20; A61M 5/14

(52) U.S. Cl. CPC A61J 1/2006; A61J 1/2065; A61M 5/14; A61M 2005/1585

(57) ABSTRACT

Infusion pump deployment systems and related methods are described. In one embodiment, after actuation, a cannulation system may simultaneously deploy a cannula of a device into tissue and release the device from the cannulation system. Associated methods and constructions of the cannulation system and devices are also described.

### CROSS REFERENCE TO RELATED APPLICATION

This application claims priority to U.S. Provisional Application No. 63/049,024, filed Jul. 7, 2020, which is hereby incorporated by reference in its entirety.

### FIELD

Disclosed embodiments are related to infusion pump deployment systems and methods.

### BACKGROUND

Ambulatory infusion pumps are oftentimes used for delivering a desired composition, such as a therapeutic compound, to a subject over long-duration periods. Depending on the particular application, ambulatory infusion pumps may either be used to deliver the desired composition subcutaneously, epidurally, and/or intravenously. Ambulatory infusion pumps are typically used for delivering these compositions to subjects where either continuous and/or repeated infusions of the composition are desired for a particular treatment. For example, certain conditions such as diabetes, cancer, chronic pain, infections, gastrointestinal conditions and others may benefit from treatments using ambulatory infusion pumps.

### SUMMARY

In one aspect, methods are provided. In some embodiments, the method of deploying an infusion pump includes positioning a device including a cannula and retained in a cannula inserter adjacent to a surface, and actuating the cannula inserter to simultaneously deploy the cannula into the surface and release the device from the cannula inserter.

In another aspect, systems are provided. In some embodiments, the system includes a device including a cannula, and a cannula inserter. The device is initially connected to the cannula inserter, and the cannula inserter is configured to retain the device prior to actuation. The cannula inserter is also configured to simultaneously deploy the cannula of the device and release the device during actuation.

In yet another aspect, cannula inserters are provided. In some embodiments, the cannula inserter includes a first lock configured to selectively connect a device including a cannula to the cannula inserter, an inserter needle configured to move between an undeployed configuration and a deployed configuration, a second lock configured to selectively restrain the inserter needle in the undeployed configuration, and a trigger. Actuation of the trigger is configured to unlock the first lock to permit the inserter needle to move from the undeployed configuration to the deployed configuration and to unlock the second lock to release the device.

In yet another aspect, infusion pumps are provided. In some embodiments, the infusion pump includes a chamber in fluid communication with one or more reservoirs, a first septum configured to seal a first portion of the chamber, an opening formed in a second portion of the chamber, a support collar configured to be inserted into and sealed against the opening in a deployed configuration, and a cannula connected to and extending from the support collar. The cannula is configured to extend through the first septum to an exterior of the infusion pump in the deployed configuration.

In yet another aspect, methods are provided. In some embodiments, the method includes deploying a cannula through a first septum of a chamber with a needle inserter to place the chamber in fluid communication with an exterior of the infusion pump, and sealing a second portion of the chamber with a second septum connected to the cannula. The needle inserter passes through the second septum and the cannula.

In yet another aspect, infusion pumps are provided. In some embodiments, the infusion pump includes a chamber in fluid communication with one or more reservoirs, a first septum configured to seal a first portion of the chamber, a second septum configured to seal a second portion of the chamber, and a cannula. The cannula is connected to and extends from the second septum into an interior of the chamber, and the cannula extends through the first septum to an exterior of the infusion pump.

In any of the embodiments above, releasing the device includes deforming two arms out of engagement with a corresponding portion of the device.

In any of the embodiments above, deforming the two arms includes deforming the two arms against a portion of a needle inserter as the needle inserter moves between an undeployed configuration in a deployed configuration to deploy the cannula.

In any of the embodiments above, actuating the cannula inserter includes camming a first lock out of engagement with a needle inserter to permit the needle inserter to move from an undeployed configuration to a deployed configuration to deploy the cannula.

In any of the embodiments above, a method includes sealing a portion of a chamber the device with a septum connected to the cannula as the cannula is deployed.

In any of the embodiments above, a method includes retracting a needle from the cannula and the device while retaining the cannula on the device.

In any of the embodiments above, deploying the cannula includes deploying an inserter needle the cannula is disposed on.

In any of the embodiments above, the device includes a septum. In some embodiments, the cannula is connected to and extends from the septum, and the septum is configured to seal at least a portion of a chamber of the device when the cannula is in a deployed configuration.

In any of the embodiments above, movement of an inserter needle of the device towards the deployed configuration releases the device from the cannula inserter.

In any of the embodiments above, movement of the inserter needle from the undeployed configuration to the deployed configuration unlocks the second lock.

In any of the embodiments above, the first lock includes two opposing arms configured to engage a corresponding portion of the device to selectively connect the device to the cannula inserter.

In any of the embodiments above, movement of the inserter needle from the undeployed configuration to the deployed configuration deforms the two opposing arms out of engagement with the corresponding portion of the device to release the device.

In any of the embodiments above, a third lock is configured to retain the cannula in the deployed configuration and connected to the device.

In any of the embodiments above, the device is an infusion pump.

In any of the embodiments above, a support collar is configured to be inserted into and sealed against an opening formed in the chamber in a deployed configuration. In some embodiments, the cannula is connected to and extends from the support collar. In some embodiments, the second septum is connected to the support collar and a proximal portion of the cannula.

In any of the embodiments above, a second septum is connected to the support collar and a proximal portion of the cannula.

In any of the embodiments above, when the support collar and the cannula are in an undeployed configuration, the support collar is distanced from the opening.

In any of the embodiments above, in an undeployed configuration the cannula extends through the first septum by a first length.

In any of the embodiments above, in the deployed configuration, the cannula extends through the first septum by a second length greater than the first length.

In any of the embodiments above, a hole is formed in a side of the cannula. In some embodiments, the hole is configured to be in fluid communication with the chamber when the cannula is in the deployed configuration.

In any of the embodiments above, a lock retains the support collar and the cannula in the deployed configuration.

In any of the embodiments above, a seal is disposed between the support collar and the opening.

In any of the embodiments above, sealing the second portion of the chamber includes inserting a support collar the cannula and the second septum are connected to into an opening formed in the second portion of the chamber.

In any of the embodiments above, the method includes retracting the needle inserter through the first and second septa while leaving the cannula in a deployed configuration.

In any of the embodiments above, the method includes deploying the cannula into a surface at an angle between about 20° and 90° relative to the surface.

In any of the embodiments above, the method includes locking the second septum in place to maintain the seal of the second portion of the chamber.

In any of the embodiments above, the first septum is pre-pierced by the cannula and needle inserter before deployment.

In any of the embodiments above, the chamber is configured to buffer a flow of liquid provided from the one or more reservoirs.

In any of the embodiments above, the cannula is configured to be deployed at an angle between about 20° and 90° relative to a portion of a subject's skin underlying the infusion pump during use.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a perspective view of one embodiment of a system including an infusion pump and a cannulation system attached to the infusion pump;

FIG. 2 is a perspective view of the system of FIG. 1 with the cannulation system detached from the infusion pump;

FIG. 3 is a perspective exploded view of one embodiment of an actuator and latches disposed within a cannulation system;

FIG. 4 is a perspective exploded view of one embodiment of a driver and associated components of a cannulation system;

FIG. 5 is a cross-sectional view of one embodiment of a needle inserter and associated springs in a preloaded compressed configuration;

FIG. 6 is a perspective view of one embodiment of a needle inserter and associated springs in a preloaded compressed configuration;

FIG. 7 is a perspective view of one embodiment of a cannula;

FIG. 8 is a cross-sectional view of one embodiment of a receiver configured to receive a cannula;

FIG. 9 is a cross-sectional view of one embodiment of a receiver configured to receive a cannula;

FIG. 10 is a perspective view of one embodiment of a system including a cannulation system and a connected infusion pump in the attached configuration;

FIG. 11 is a perspective cross-sectional view of the system of FIG. 10;

FIG. 12A is a perspective view of one embodiment of a cannulation system and infusion pump in a pre-deployed configuration;

FIG. 12B is a close-up perspective view of the system of FIG. 12A with components removed to illustrate the interface between the cannulation system and infusion pump;

FIG. 12C is a cross-sectional view of the system of FIG. 12A;

FIG. 13 is a cross-sectional view of one embodiment of a cannulation system and infusion pump with a needle inserter partially deployed;

FIG. 14 is a cross-sectional view of one embodiment of an actuation button and associated holder that are configured to selectively actuate a cannulation system

FIG. 15 is a cross-sectional view of one embodiment of a cannulation system with the needle inserter and cannula in the deployed configuration;

FIG. 16 is a cross-sectional view of one embodiment of the retraction mechanism of a cannulation system after cannula deployment;

FIGS. 17A-17C depict one embodiment of a cannulation system and infusion pump with the cannula and needle inserter in the deployed configuration;

FIGS. 18A-18B depict one embodiment of an infusion pump after removal of a cannulation system and associated needle inserter;

FIG. 19 depicts one embodiment of a system including a cannulation system and associated disposable module;

FIG. 20 depicts one embodiment of an infusion pump with a selectively connectable electronics module; and

FIG. 21 depicts the infusion pump and connectable electronics module of FIG. 20 in the assembled configuration.

### DETAILED DESCRIPTION

Typical cannulation systems for use with infusion pumps and other devices typically require a user to separately assemble, attach, actuate, and subsequently release the various components of the devices during a cannulation procedure. The Inventors have recognized that these types of operations may lead to user errors, are lengthy, and may result in increased operational difficulty when such systems are applied by an end user.

In view of the above, the Inventors have recognized the benefits associated with systems including a cannulation system that simultaneously releases a device and deploys a cannula of the device into the tissue of a user after actuation of the cannulation system by a user. For example, a user may actuate a trigger of the cannulation system which may release a needle inserter of the cannulation system and actuate a lock of the cannulation system to release the associated device which may be an infusion pump. Such a system may simplify user operation while also helping to avoid errors during deployment of a cannula into tissue. Associated methods and constructions are described in further detail below. However, embodiments in which the various systems and devices subscribed herein are operated without simultaneously deploying a cannula and releasing an associated device are also contemplated as the disclosure is not limited in this fashion.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

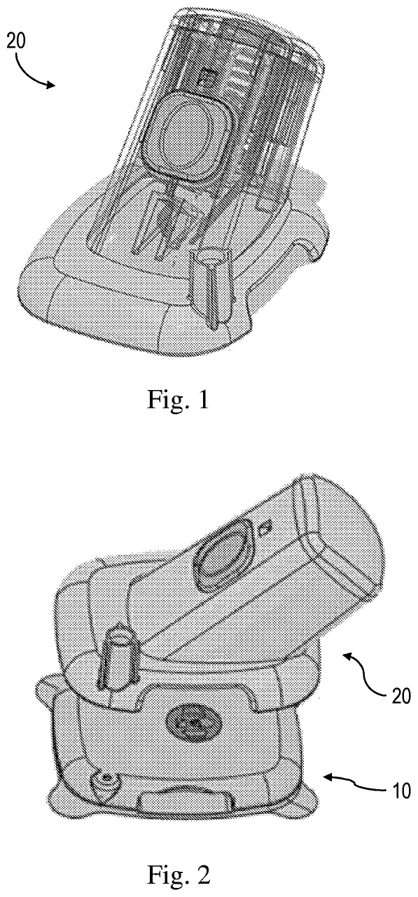

FIGS. 1 and 2 depict one embodiment of a system that includes a disposable module 10 in the form of an infusion pump that may be attached to and subsequently released from a cannulation system 20 during a cannula deployment operation. As described in further detail below. In some embodiments, the cannulation system and the disposable module may include aligned filling ports to facilitate filling of the disposable module with one or more therapeutic compounds for subsequent deployment into the tissue of a user.

In some embodiments, a cannulation system may include 11 individual components and 3 main subassemblies. The elements of a cannulation system may include a: base subassembly with left and right latches, an actuator, and a base; and a cover subassembly including a cannula subassembly, a needle inserter, a driver, holder, an insertion spring, a retraction spring, and a top. Various embodiments of these different components are described in further detail below.

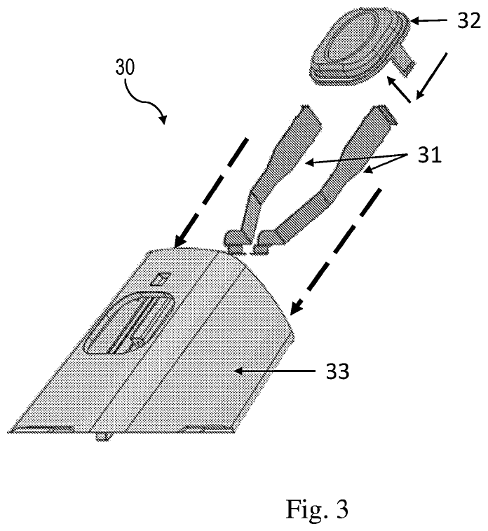

As shown in FIG. 3, a base subassembly 30 may include three primary components in the form of two latches 31, a button or actuator 32, and a base 33. The latches 31 and the button 32 may be press fit into the base 33 in some embodiments. This subassembly 30 may latch onto a receiver on a disposable module of an infusion pump and provide alignment for the cannula stroke as well as an interface for the user to deploy the cannula into the subcutaneous tissue.

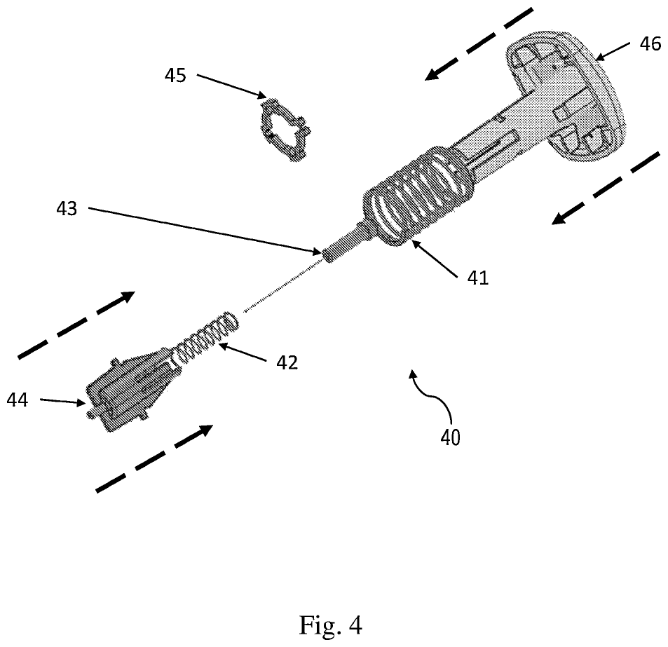

FIG. 4 depicts one embodiment of a cover subassembly 40. In the depicted embodiment, the cover subassembly 40 includes: two springs 41,42 (one for insertion 41 and one for retraction 42 of a needle inserter), the needle inserter, 43 a driver 44, a holder 45, a cover 46, and a cannula subassembly. Construction of the cover subassembly 40 may be considered independent of the cannula subassembly, which may be introduced at any point during the cover assembly process.

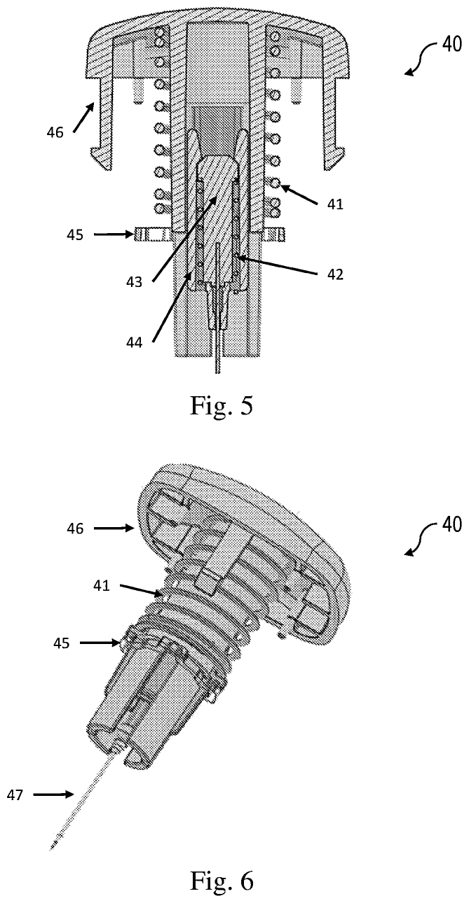

As shown in FIGS. 5 and 6, in one embodiment a cover subassembly 40, a cannulation system may include two compression springs to separately introduce the cannula and needle inserter into the subcutaneous tissue (insertion spring 41), and retract the needle inserter from the tissue once the cannula has locked into the receiver (retraction spring 42). The springs 41,42 may be preloaded during storage and energy may be released only when the actuator or button is pressed by the user. As seen below, the needle inserter 43 and the retraction spring 42 are housed inside the driver 44 prior to deployment. A tight fit between the cover 46 and the driver 44 may prevent the driver's arms from opening and allowing the compressed retraction spring 42 to extend. During assembly, the driver 44, retraction spring 42, and needle inserter 43 slide into the cover 46 and may be pushed towards the top of the cover 46 to preload the insertion spring 41. Once the insertion spring 41 is fully compressed to its pre-deployment position, the holder 45 is snapped into position which locks the driver 44 into a pre-deployment position. Finally, the cannula sub-assembly 47 may be slid into place.

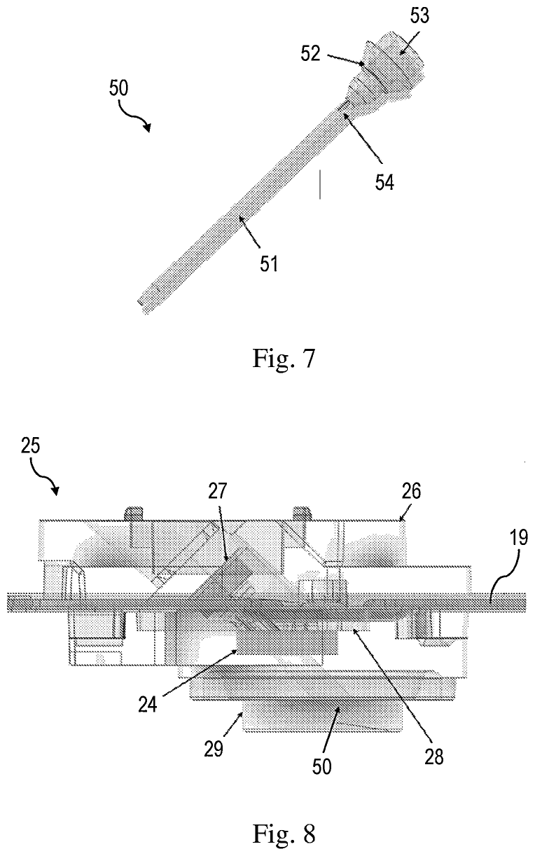

FIG. 7 depicts one embodiment of a cannula 50 which includes three parts: a tube 51, a support column 52, and a septum 53. The polymeric tube 51 starts as an extruded low-diameter, low-wall-thickness tube with an inner diameter intended to house a needle that will guide and provide support for the cannula 50 as the needle and cannula penetrate the skin of a subject and/or elastomeric seals, such as septums, of an infusion pump or other device. The cannula tube 51 may be cut to an appropriate length and subjected to tipping and flanging processes (thermal forming process) which may optimize the tube's geometry for skin penetration and attachment to a support structure, respectively. The cannula tube 51 may also include a side port 54, in the form of an opening, that is cut (e.g. laser-ablation) or otherwise formed in a portion of a side of the cannula 50 in a region that is in fluid communication with one or more reservoirs of the infusion pump in a deployed position. Thus, the cannula 50 may provide a flow path from the infusion pump to the subcutaneous tissue of a subject for flowing an active pharmaceutical ingredient (API), or other therapeutic compounds, to the tissue of a subject. In embodiments including a support, such as the depicted support column, the support may be an injection molded polymer structure (typically a thermoplastic that can be bonded to the tube) though other manufacturing methods and materials may also be used. The support column 52 may be bonded to the tube 51 and may be configured to establish two seals including: a first seal with a flanged side of the cannula tube; and a second seal with an open port of the infusion pump that the support column is pressed against in a deployed configuration. The cannula's septum 53 may be housed by the support column 52 and may be made from a medical-grade silicone or other appropriate elastic or elastomeric material capable of sealing the cannula 50. Thus, the cannula's septum may enable the use of a needle-inserter during cannula deployment without leakage post-deployment.

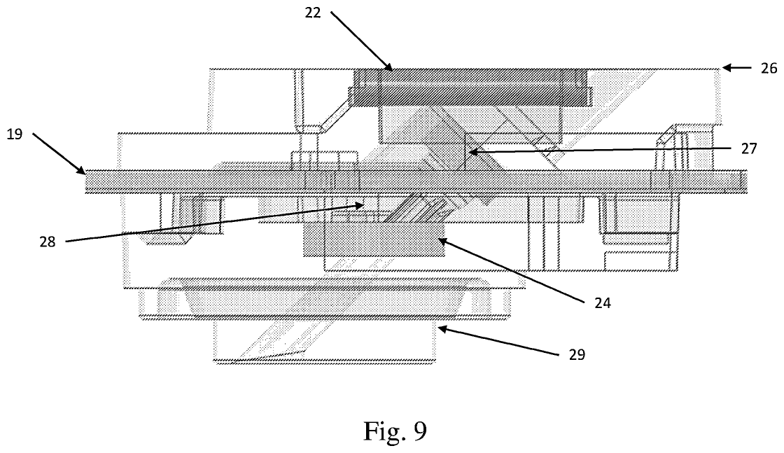

FIGS. 8 and 9 depict one embodiment of a receiving structure including an outlet 25 of an infusion pump that is configured to accept a cannula 50 during deployment of a cannula into tissue. The depicted outlet includes five pieces: the receiver 26, the receiver's septum 27, the distributor 28, the base 29, and the base's septum 24. The outlet 25 is thermally sealed to the microfluidic laminates 19 and mechanically sealed to the cannula 50. Initially, the injection molded distributor 28 and base 29 may compress the base's septum 24. This compression may be made permanent by laminating the distributor 28 and the base 29 to the microfluidic laminates 19. Separately, the receiver's septum 27 may be placed in the distributor 28 and the receiver 26 may be ultrasonically welded, or otherwise attached, to the base 29, which may fix a desired compression level of the receiver's septum 27 and the alignment of all parts of the inserter (including the cannula 50) and the outlet 25. The receiver's septum 27 is not intended to be pierced but instead behaves more like an o-ring when pressed against the support column, thus, in some embodiments, the receiver's septum 27 may also be referred to as a seal in some embodiments.

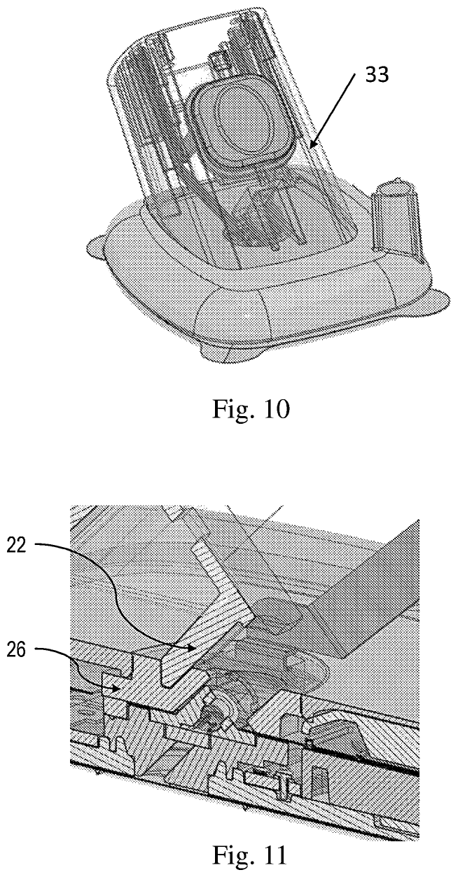

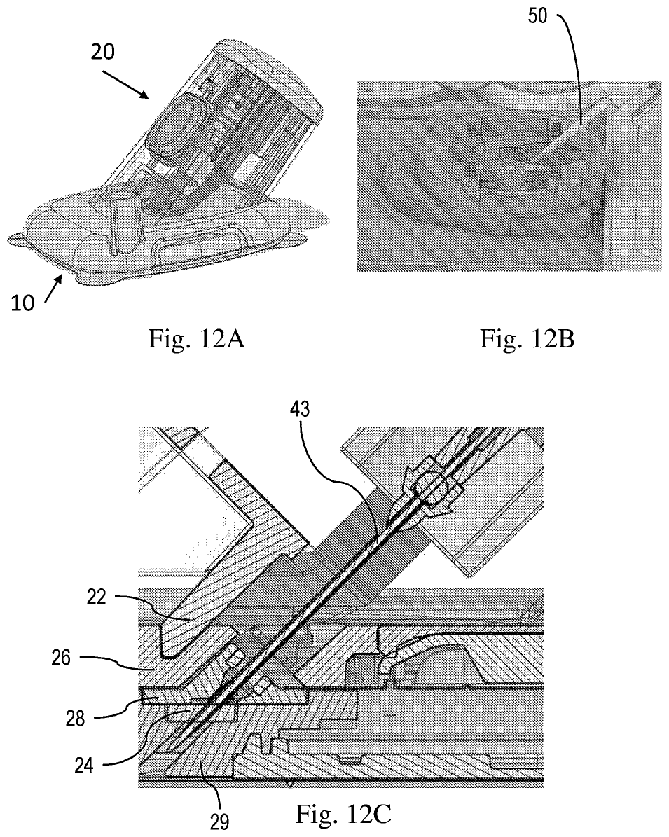

Once a device is fully assembled, the inserter base may be locked onto the receiver 26 by snapping the latches into place with one or more posts 22, indentations, or other mechanically interlocking features located on the receiver 26, see FIGS. 10-11. This may provide alignment for the inserter cover to slide into place with the help of alignment features on the inserter base, see FIGS. 12A-12C.

Still referring to FIGS. 12A-12C, once a full assembly is complete, the deployment system remains firmly attached to the disposable device 10 through the engagement of the latches on the receiver 26. Pre-deployment the cannula 50 may be prepositioned 1 mm-1.5 mm, or any other appropriate distance, from the skin. A needle-inserter 43, typically a stainless-steel stylet, is placed inside the cannula 50. The cannula 50 and the needle-inserter 43 pierce through the base's septum 24. By having the cannula and needle-inserter in this position before deployment, the piercing of the cannula's septum and the base's septum may be controlled during a manufacturing process of the device, though instances in which a needle inserter is not prepierced into a septum of a device are also contemplated. In the depicted predeployment configuration, there is no seal between the support column 52 and the receiver's septum 27, which may help facilitate a filling process of the device by allowing air to be displaced from the inlet of the fluid path towards the outlet of the fluid path.

During actuation of the deployment system, the inserter will drive the cannula 50 and needle-inserter 43 into the tissue. To the user's perspective, the insertion cycle may appear to be instantaneous. However, in some embodiments three distinct stages of deployment may occur during operation once the user presses the actuator.

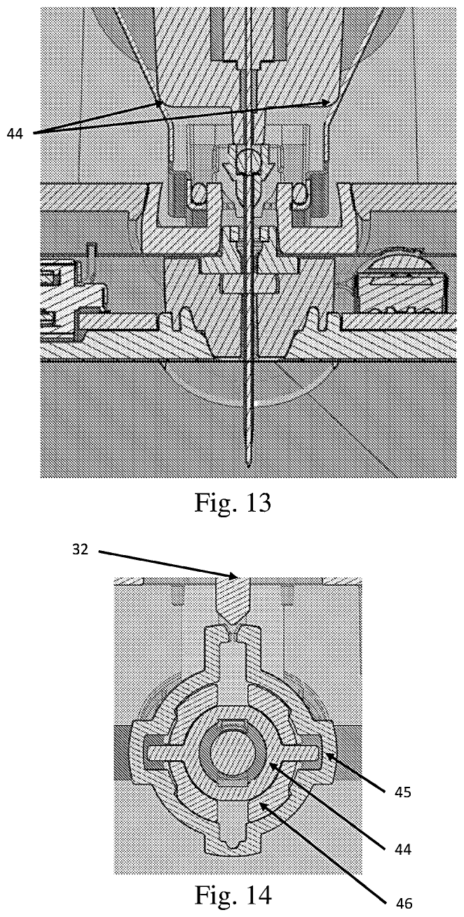

As shown in FIGS. 13-14, in one embodiment, a first deployment stage may primarily include the holder 45 release. During this initial stage, the user overcomes a threshold force to disengage the holder from the applicator's cover. When the holder 45 opens, the pre-loaded insertion spring 41 will transform its potential energy into kinetic energy, which will push the driver 44, needle inserter 43, cannula subassembly, and release spring 42 towards the skin (see FIG. 4 for more details). The impulse from the insertion spring 41 onto the driver 44 may be large enough to provide sufficient momentum to overcome any restraining forces from the latches and the receiver snap points applied to the driver 44 and the cannula 50.

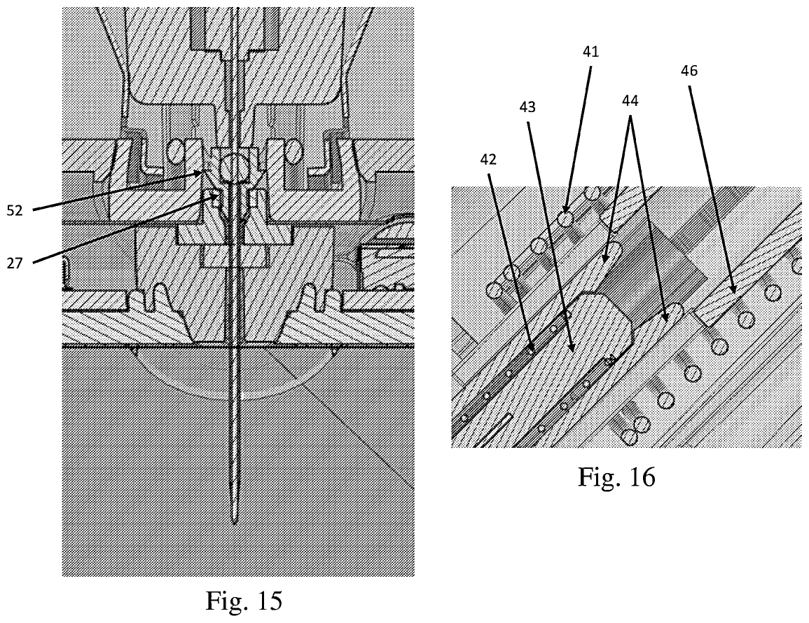

During a second stage the cannula may lock into its final deployed position and the deployment system may release the infusion pump, see FIGS. 15-16. At this stage, the hydraulic channel between the reservoirs and the subcutaneous tissue is complete. The driver 44 is no longer radially suppressed by the applicator cover 46 and will allow potential energy from the retraction spring 42 to be released. In the deployed configuration, the support column 52 is radially compressed against the receiver's septum 27 which is in the form of a seal extending around the perimeter of the support collar to seal the associated opening. The seal may be made permanent by using a latching mechanism that prevents the support column from being displaced relative to the receiver. In this state, the outlet is completely sealed and there is no fluid path for the therapeutic compound to access the tissue even though the cannula sits in its final position.

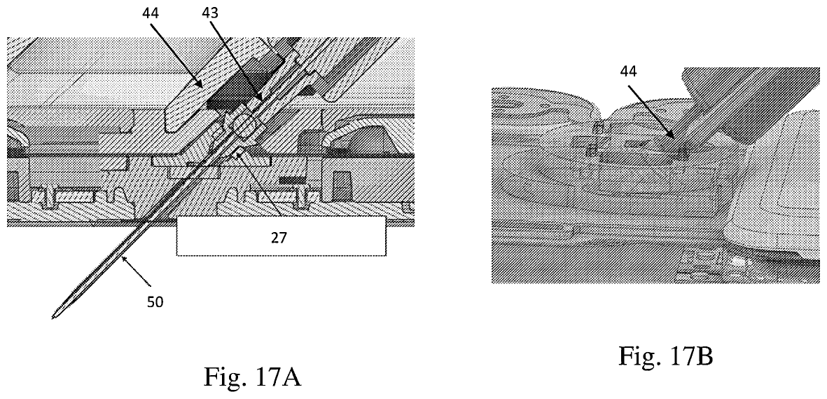

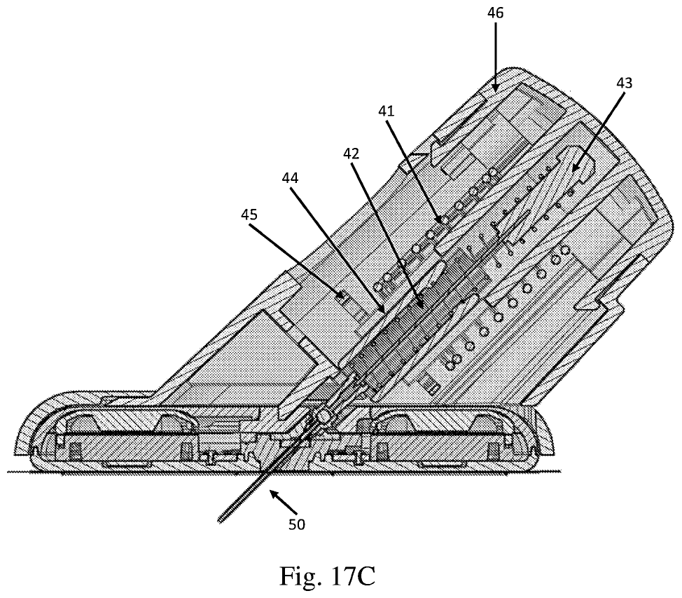

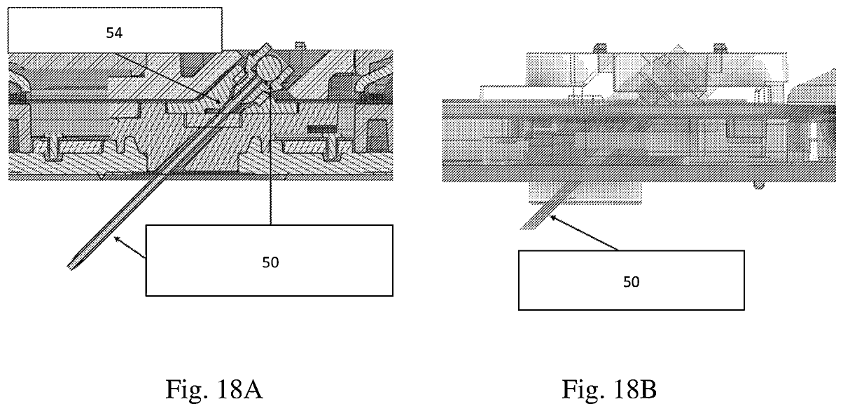

During a third final stage of deployment, the retraction spring, which sits preloaded inside the driver prior to cannula deployment (see FIG. 5), may be free to displace the needle inserter 43 in the opposite direction along the insertion plane towards the cover of the applicator, see FIGS. 17A-17C. This removes the needle inserter 43 from within the deployed cannula while the cannula 50 remains locked in the deployed configuration due to the cannula support being locked to the receiver 26 of the infusion pump. In its final position, the needle inserter's sharp tip may be hidden inside the driver 44, protecting the user from accidental puncture when handling the deployment system, see FIGS. 18A-18B.

During operation of the infusion pump to deliver a therapeutic compound to the tissue of a subject, the needle-retraction process has taken place and there is a clear path from the microfluidic laminates, through the distributor (distributes a liquid including a therapeutic compound), through the side-port on the cannula, and into the tissue through a distal end of the cannula deployed into the subject's tissue. As shown in the figures, the cannula septum seals the needle track left from the needle-inserter while providing a flow path from the one or more reservoirs of an infusion pump to the tissue.

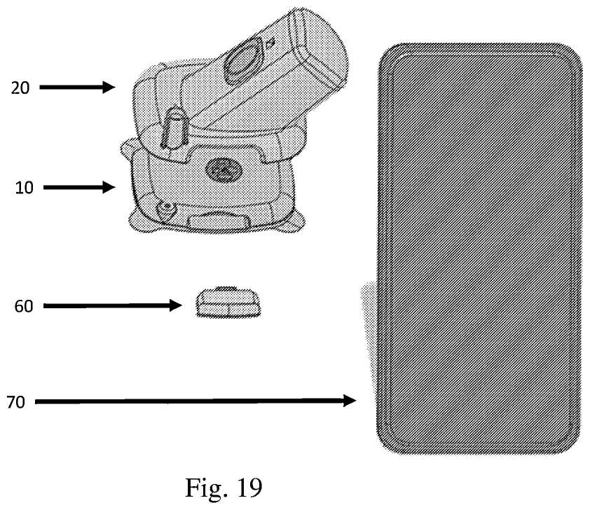

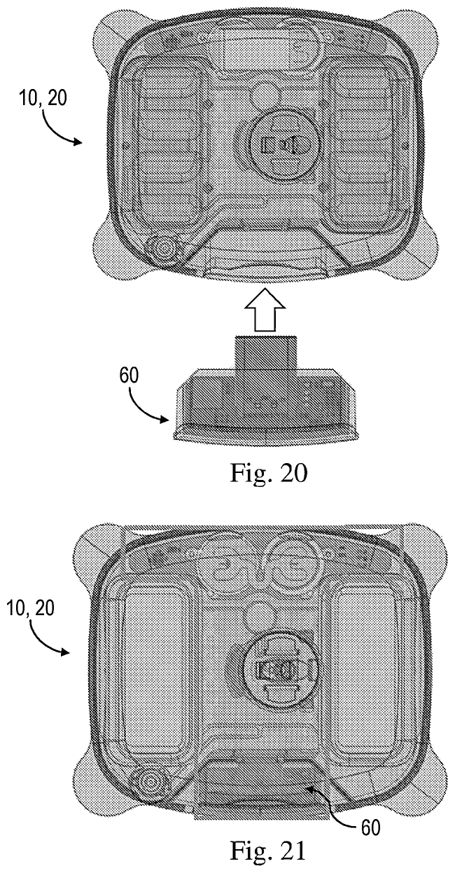

In some embodiments, a cannula deployment system may include an ambulatory infusion pump 10 that may be attached to a user's skin for subcutaneous delivery of a therapeutic compound. As depicted in FIG. 19-21, a system may include a disposable module 10 or cartridge, a reusable electronics module 60, a cannulation system 20, and a controller 70. The disposable module 10 may include the fluid path for the therapeutic compound as well as one or more actuators, such as an electrochemical actuator, that will drive the therapeutic compound into the tissue. Attached to the base of the disposable module 10 there may be a breathable adhesive to secure the device to the user's skin. The disposable module 10 may include an electrical connector that selectively receives and interfaces with the reusable electronics module 60.

In the depicted embodiment, most of the integrated circuit components may be housed in the reusable electronics module 60. The module 60 may interface with the one or more actuators contained within the disposable module 10 and a separate controller which in the depicted embodiment is a handheld computing device such as a tablet or smart phone though any other appropriate computing device including a stationary computer may be used as the disclosure is not limited in this fashion. A processor of the reusable electronics module 60 may be programmed from a user interface of the controller 70. The processor of the reusable electronics module 60 may also be configured to gather data that may then be uploaded to the controller 70. The controller 70 may be configured to present a user interface for controlling operation of the infusion pump 10. Through this user interface, users and/or caregivers may be able to establish dosage parameters as well as access data sent back through the reusable electronics module.

### EXAMPLE

### Sample Operation

Prior to use of a device, the user or caregiver may input a dosing schedule for the pump to be used through the controller. Operation of the device may include the following:
1. User opens new package with disposable module and preassembled cannulation system;
2. User connects the reusable electronics module to the disposable module;
3. Controller and reusable electronics module may signal that device is ready to be filled;
4. User fills device through port on cannulation system;
5. User visually confirms that device has been properly filled;
6. User removes adhesive liner and adheres the device on the desired area of the skin for delivery;
7. User presses button on cannulation system and separates cannulation system from patch pump;
8. User confirms successful cannulation on controller's user interface and dosing schedule begins;
9. At the time the dosing schedule begins, the reusable electronics module and the disposable module may complete a full circuit that is used to control operation of the pump, sense changes in the behavior of the pump, and alarm the user of any problems with the device; and
10. A dosage delivered by the infusion pump may be controlled through a circuit design that accurately regulates the level of electrical current flowing through any set of electrodes within an electrolytic chamber and the duration of such electrical signal (typically a square-wave). A compensation algorithm is used to account for potential changes in gas volume within each electrolytic engine from variations in atmospheric conditions (temperature and pressure).

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of deploying an infusion pump, the method comprising:
positioning a device including a cannula and retained in a cannula inserter adjacent to a surface; and
actuating the cannula inserter to simultaneously deploy the cannula into the surface and release the device from the cannula inserter.

2. The method of claim 1, wherein releasing the device includes deforming two arms out of engagement with a corresponding portion of the device.

3. The method of claim 2, wherein deforming the two arms includes deforming the two arms against a portion of a needle inserter as the needle inserter moves between an undeployed configuration in a deployed configuration to deploy the cannula.

4. The method of claim 1, wherein actuating the cannula inserter includes camming a first lock out of engagement with a needle inserter to permit the needle inserter to move from an undeployed configuration to a deployed configuration to deploy the cannula.

5. The method of claim 1, further comprising sealing a portion of a chamber the device with a septum connected to the cannula as the cannula is deployed.

6. The method of claim 1, further comprising retracting a needle from the cannula and the device while retaining the cannula on the device.

7. The method of claim 1, wherein deploying the cannula includes deploying an inserter needle the cannula is disposed on.

8. A system comprising:
a device including a cannula; and
a cannula inserter, wherein the device is initially connected to the cannula inserter, and wherein the cannula inserter is configured to retain the device prior to actuation, and wherein the cannula inserter is configured to simultaneously deploy the cannula of the device and release the device during actuation.

9. The system of claim 8, wherein the device further comprises a septum, wherein the cannula is connected to and extends from the septum, and wherein the septum is configured to seal at least a portion of a chamber of the device when the cannula is in a deployed configuration.

10. The system of claim 9, wherein movement of an inserter needle of the device towards the deployed configuration releases the device from the cannula inserter.

11. A cannula inserter comprising:
a first lock configured to selectively connect a device including a cannula to the cannula inserter;
an inserter needle configured to move between an undeployed configuration and a deployed configuration;
a second lock configured to selectively restrain the inserter needle in the undeployed configuration; and
a trigger, wherein actuation of the trigger is configured to unlock the first lock to permit the inserter needle to move from the undeployed configuration to the deployed configuration and to unlock the second lock to release the device.

12. The cannula inserter of claim 11, wherein movement of the inserter needle from the undeployed configuration to the deployed configuration unlocks the second lock.

13. The cannula inserter of claim 11, wherein the first lock comprises two opposing arms configured to engage a corresponding portion of the device to selectively connect the device to the cannula inserter.

14. The cannula inserter of claim 13, wherein movement of the inserter needle from the undeployed configuration to the deployed configuration deforms the two opposing arms out of engagement with the corresponding portion of the device to release the device.

15. The cannula inserter of claim 11, further comprising a third lock configured to retain the cannula in the deployed configuration and connected to the device.

16. The cannula inserter of claim 11, wherein the device is an infusion pump.

17-34. (canceled)

35. The system of claim 8, further comprising a first lock and a second lock, wherein the first lock is configured to initially connect the device to the cannula inserter, and wherein the second lock is configured to restrain an inserter needle in an undeployed configuration.

36. The system of claim 35, further comprising a trigger, wherein the trigger is configured to unlock the first lock to permit the inserter needle to move from the undeployed configuration to the deployed configuration, and wherein movement of the inserter needle towards the deployed configuration is configured to unlock the second lock to release the device.

37. The system of claim 8, further comprising a third lock configured to retain the cannula in the deployed configuration and connected to the device.

38. The system of claim 8, wherein the device is an infusion pump.
